(19) United States
(12) Patent Application Publication
(10) Pub. No.: US 2019/0082984 A1
(43) Pub. Date: Mar. 21, 2019

(54) METHOD AND ELECTRONIC DEVICE CAPABLE OF ESTABLISHING PERSONAL BLOOD PRESSURE ESTIMATION MODEL FOR SPECIFIC USER/PERSON

(71) Applicant: PixArt Imaging Inc., Hsin-Chu City (TW)

(72) Inventors: Wei-Ru Han, Hsin-Chu City (TW); Chih-Yuan Chuang, Hsin-Chu City (TW); Yuan-Hsin Liao, Hsin-Chu City (TW)

(21) Appl. No.: 15/709,458

(22) Filed: Sep. 19, 2017

(51) Int. Cl. A61B 5/024; A61B 5/00

(52) U.S. Cl. CPC A61B 5/02416; A61B 5/7221; A61B 5/7235

(57) ABSTRACT

A method for establishing a personal blood pressure estimation model dedicated for a specific user includes: receiving a first reference measurement result of a reference sphygmomanometer; using a PPG sensor to measure a blood pressure of the specific user to generate a first PPG signal; calculating a first estimation result of the blood pressure of the specific user according to the first PPG signal; generating a first regulating parameter by comparing the first reference measurement result with the first estimation result; and establishing the personal blood pressure estimation model by using the first regulating parameter to adjust a set of parameter factor (s) of a basic blood pressure estimation model.

### BACKGROUND OF THE INVENTION

### 1. Field of the Invention

The invention relates to a blood pressure estimation mechanism, and more particularly to a method and an electronic device such as an interactive human-machine interface device capable of establishing a personal blood pressure estimation model for a specific user/person.

### 2. Description of the Prior Art

Generally speaking, a conventional blood pressure estimation scheme may be arranged to detect a user's physiological characteristics and calculate a static blood pressure estimation result based on the detected physiological characteristics and a basic or general blood pressure estimation model such as an estimation curve. However, the blood pressure estimation is not like the heart rate estimation. The change of a user's blood pressure is significantly different from that of another user's blood pressure. In addition, the change of static blood pressure is significantly different from that of dynamic blood pressure. It is possible to use the basic or general blood pressure estimation model to obtain/generate a blood pressure estimation result, but it is impossible to make blood pressure estimation results more accurate for many users by merely using the same basic or general blood pressure estimation model to estimate blood pressure.

### SUMMARY OF THE INVENTION

Therefore one of the objectives of the invention is to provide a method and an electronic device of establishing or generating a personal (static and/or dynamic) blood pressure estimation model dedicated for a specific person or user based on a basic/general blood pressure estimation model, to solve the above-mentioned problems. The device can generate personal blood pressure estimation models for different persons or users based on the same basic/general blood pressure estimation model.

According to embodiments of the invention, a method for establishing a personal blood pressure estimation model dedicated for a specific user is disclosed. The method comprises: receiving a first reference measurement result of a reference sphygmomanometer; using a photoplethysmogram sensor to measure a blood pressure of the specific user to generate a first photoplethysmogram (PPG) signal; calculating a first estimation result of the blood pressure of the specific user according to the first PPG signal; generating a first regulating parameter by comparing the first reference measurement result with the first estimation result; and establishing the personal blood pressure estimation model by using the first regulating parameter to adjust a set of parameter factor(s) of a basic blood pressure estimation model.

According to the embodiments, an electronic device for establishing a personal blood pressure estimation model dedicated for a specific user is disclosed. The electronic device comprises a receiving unit, a PPG sensor, and a processing circuit. The receiving unit is configured to receive a first reference measurement result of a reference sphygmomanometer. The PPG sensor is configured to measure a blood pressure of the specific user to generate a first PPG signal. The processing circuit is coupled to the receiving unit and the PPG sensor, and is configured to: calculate a first estimation result of the blood pressure of the specific user according to the first PPG signal; generate a first regulating parameter by comparing the first reference measurement result with the first estimation result; and establish the personal blood pressure estimation model by using the first regulating parameter to adjust a set of parameter factor(s) of a basic blood pressure estimation model.

In addition, the electronic device for example is an interactive human-machine interface device.

In addition, the electronic device incorporating the method can generate the personal static and dynamic blood pressure estimation models dedicated for a specific user according to a basic estimation model, two reference measurement results of an external sphygmomanometer respectively corresponding to static and dynamic blood pressures, and two PPG signals respectively corresponding to static and dynamic blood pressures. It is convenient for a user to operate the electronic device. For example, when the electronic device is initially activated or is arranged to regularly update the models, a user may be asked to measure blood pressure by using the external sphygmomanometer two times, and other operations can be performed by the electronic device automatically. In addition, if the user has a habit of operating the external sphygmomanometer, the user may not be asked to measure blood pressure. The electronic device can directly receive the reference measurement results from the external sphygmomanometer via wired/wireless communication, and all operations can be performed or executed by the electronic device itself. That is, the user hardly feels the influence on operating the external sphygmomanometer in the personal estimation model establish process of the electronic device. This brings a good experience for users.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram showing a flowchart of a method used for establishing or generating a personal blood pressure estimation model of a specific user/person according to embodiments of the invention.

FIG. 2 is a diagram illustrating an electronic device used for establishing or generating the personal blood pressure estimation model of the specific user/person according to the embodiment flowchart of FIG. 1.

FIG. 3 is a diagram showing an example of one PPG waveform signal generated by the PPG sensor of electronic device of FIG. 2.

### DETAILED DESCRIPTION

The embodiments of the invention aim to provide a method which is capable of establishing personal physiological characteristics estimation models for different persons/users, i.e. to provide/generate different estimation models respectively dedicated for different persons. The method is arranged to calibrate or adjust parameter factor(s) of a preliminary/preset/basic physiological characteristics estimation model (e.g. estimation curve) by referring to a user's physiological characteristics result(s) measured by a photoplethysmogram (PPG) sensor and the user's physiological characteristics result(s) measured by a reference physiological characteristics detection device, to generate and provide a personal physiological characteristics estimation model dedicated to such user. Thus, the physiological characteristics of the user can be precisely and accurately estimated by using such personal physiological characteristics estimation model of the method.

For example, the physiological characteristics mean the user's blood pressure. The preliminary/preset/basic physiological characteristics estimation model means a preliminary/preset/basic blood pressure estimation model, and the personal physiological characteristics estimation model means a personal/dedicated blood pressure estimation model for a particular/specific user. The reference physiological characteristics detection device may be a reference sphygmomanometer (meter or monitor); however, this is not intended to be a limitation. The sphygmomanometer may be implemented using various kinds of sphygmomanometers which may be produced by different manufacturers such as Omron Healthcare Company or other manufacturers.

In the embodiments, the reference sphygmomanometer indicates a larger electronic device which can generate reference/accurate measurement result(s) for the blood pressure of the user; the reference sphygmomanometer is not like a mobile device, a handheld device or a wearable electronic device. The method is to provide the PPG sensor with the basic blood pressure estimation model, use the PPG sensor with the basic blood pressure estimation model to obtain estimation result(s) of the blood pressure of the user, and finally to fix/calibrate/adjust parameter factor(s) of such basic estimation model based on the reference/accurate measurement result(s) and the obtained estimation result(s) to generate the personal or dedicated blood pressure estimation model.

It should be noted that, to obtain precise/accurate parameter factor(s) of personal or dedicated blood pressure estimation model, the method may be arranged to ask the user to operate the reference sphygmomanometer and the PPG sensor based on identical/similar user behaviors so as to obtain more accurate/precise parameter factor(s) as far as possible. For instance, the method may be arranged to ask the user to operate the reference sphygmomanometer and the PPG sensor after he or she has took a rest. However, this is not meant to be a limitation.

In addition, the same type of device incorporating the above method may be operated by different users, even though the parameter factor(s) of the basic estimation model is/are identical, the method can be arranged to adjust the parameter factor(s) as different parameter factor(s) based on different users' physiological characteristics to generate different personal estimation models respectively dedicated to different persons.

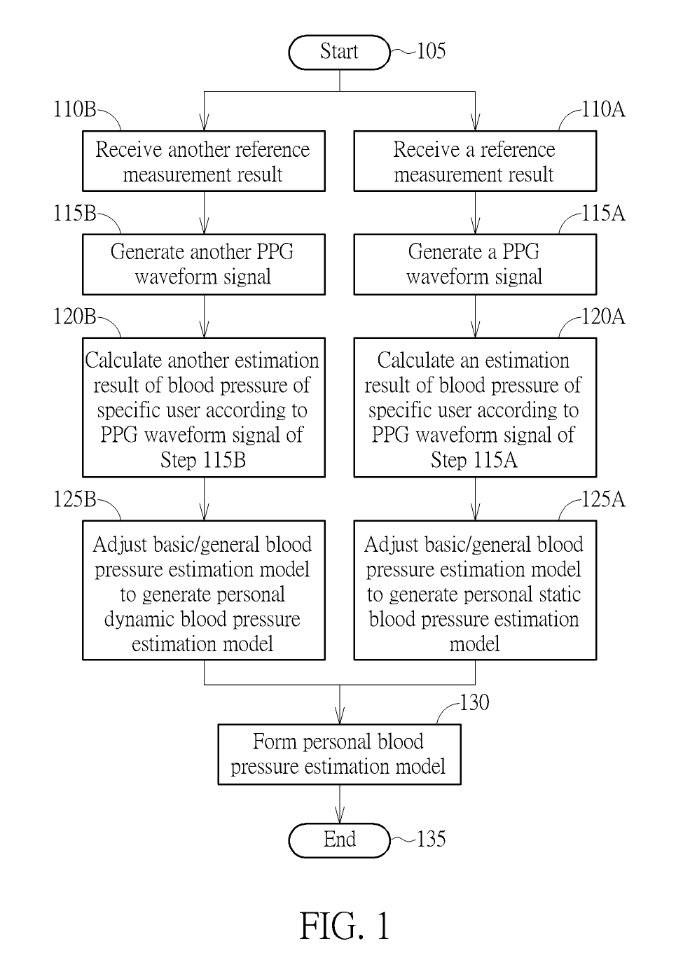

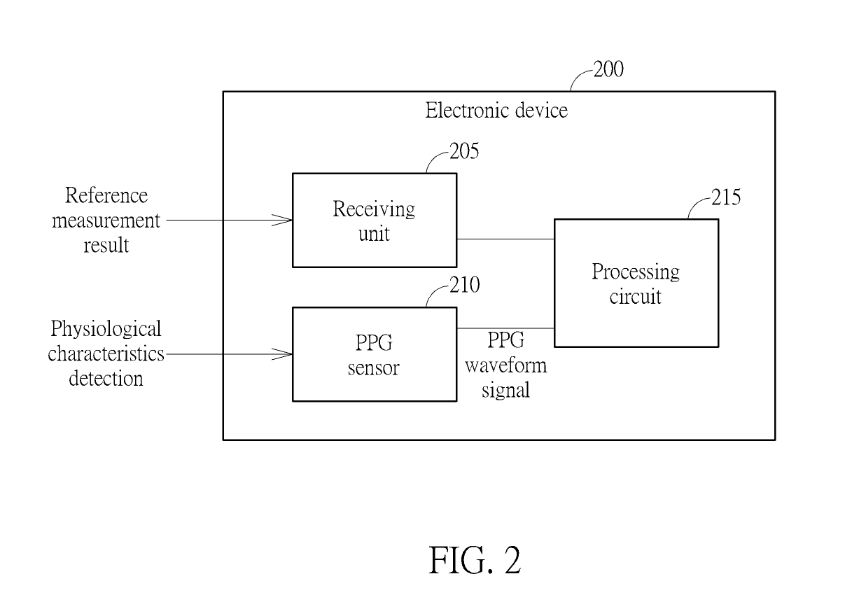

Refer to FIG. 1 in conjunction with FIG. 2. FIG. 1 is a diagram showing a flowchart of a method used for establishing or generating a personal blood pressure estimation model of a specific user/person according to embodiments of the invention. FIG. 2 is a diagram illustrating an electronic device 200 used for establishing or generating the personal blood pressure estimation model of the specific user/person according to the embodiment flowchart of FIG. 1. The electronic device 200 such as a mobile device, a handheld device, a wearable electronic device, or an interactive human-machine interface device, and comprises a receiving unit 205 (hardware circuit, software element, or combinations), a PPG sensor 210, and a processing circuit 215 such as a processor; the reference sphygmomanometer is not shown on FIG. 2. The electronic device 200 is capable of establishing or generating a personal blood pressure estimation model of a specific user based on a basic/general blood pressure estimation model and then using the personal blood pressure estimation model to measure/detect the specific user's blood pressure to obtain more accurate blood pressure information. Provided that substantially the same result is achieved, the steps of the flowchart shown in FIG. 1 need not be in the exact order shown and need not be contiguous, that is, other steps can be intermediate. Steps are detailed in the following:

Step 105: Start;

Step 110A: Use the receiving unit 205 to receive a reference measurement result of the reference sphygmomanometer wherein the reference measurement result is obtained when the change of blood pressure of specific user is at a linear state;

Step 110B: Use the receiving unit 205 to receive another reference measurement result of the reference sphygmomanometer wherein the another reference measurement result is obtained when the change of blood pressure of specific user is at a non-linear state;

Step 115A: Use the PPG sensor 210 to detect the physiological characteristics of the specific user for one time to generate a PPG waveform signal when the change of blood pressure of specific user is at the linear state;

Step 115B: Use the PPG sensor 210 to detect the physiological characteristics of the specific user for one time to generate a PPG waveform signal when the change of blood pressure of specific user is at the non-linear state;

Step 120A: Use the processing circuit 215 to calculate and obtain an estimation result of the blood pressure of the specific user according to the generated the PPG waveform signal of Step 115A;

Step 120B: Use the processing circuit 215 to calculate and obtain another estimation result of the blood pressure of the specific user according to the generated the PPG waveform signal of Step 115B;

Step 125A: Adjust a set of parameter factor(s) of the basic/general blood pressure estimation model used by the PPG sensor 210 by comparing the reference measurement result of Step 110A with the estimation result of blood pressure of Step 120A, to generate the personal static blood pressure estimation model dedicated for the specific user;

Step 125B: Adjust another set of parameter factor(s) of the basic/general blood pressure estimation model used by the PPG sensor 210 by comparing the reference measurement result of Step 110B with the estimation result of blood pressure of Step 120B, to generate the personal dynamic blood pressure estimation model dedicated for the specific user;

Step 130: Generate the personal blood pressure estimation model based on the personal static blood pressure estimation model and personal dynamic blood pressure estimation model; and

Step 135: End.

In the embodiments, a reference measurement result has a reference value of systolic blood pressure and a reference value of diastolic blood pressure, and an estimation result of blood pressure has an estimation value of systolic blood pressure and an estimation value of diastolic blood pressure. That is, the electronic device 200 incorporating the method is arranged to estimate the specific user's systolic blood pressure and diastolic blood pressure. This is not intended to be a limitation. In another embodiment, the electronic device 200 may be arranged to estimate one of the systolic blood pressure and diastolic blood pressure.

Additionally, the change of blood pressure of a specific user may be at a steady state and at a non-steady state respectively corresponds to different behaviors/actions of the specific user. For example, the steady state may mean that the specific user takes a rest for a few minutes. The non-steady state has two different possible states comprising the above-mentioned linear state and non-linear state. The linear state means that the values of specific user's systolic and diastolic blood pressures are linearly changed with the heart rate of the specific user, and the non-linear state means that the values of specific user's systolic and diastolic blood pressures are non-linearly changed with the heart rate of the specific user.

For example, in a default setting of the device 200, the linear state may mean that the specific user participates in daily activities such as brushing teeth and washing face (but not limited) and/or indoor activities (excluding exercise) such as playing cards or walking around a room slowly, and so on. The linear state is associated with the specific user's static blood pressure estimation. In the default setting, the non-linear state may mean that the specific user participates in indoor exercise activities (e.g. dancing, rock climbing, or others) and/or outdoor activities such as exercising, jogging, or catching a bus, and so on. The non-linear state is associated with the specific user's dynamic blood pressure estimation. Generating the personal blood pressure estimation model dedicated for the specific user comprises generating a personal static blood pressure estimation model and/or a personal dynamic blood pressure estimation model.

For generating the personal static blood pressure estimation model and the personal dynamic blood pressure estimation model, in Step 110A and Step 110B, the electronic device 200 is arranged to control the receiving unit 205 to at least receive two reference measurement results which are measured and obtained by the reference sphygmomanometer when the change of blood pressure of specific user is at the linear state and non-linear state respectively.

Correspondingly, in Step 115A and Step 115B, the electronic device 200 is arranged to control and use the PPG sensor 210 to detect the physiological characteristics of the specific user for two times to generate two PPG waveform signals when the change of blood pressure of specific user is at the linear state and non-linear state respectively.

If the reference measurement result received by the receiving unit 205 is measured and obtained when the change of blood pressure of specific user is at the linear state, the PPG sensor 210 is activated to detect the physiological characteristics of the specific user for one time to generate one PPG waveform signal when the change of blood pressure of specific user is at the same linear state, so that the processing circuit 215 can adjust corresponding parameter factor (s) of the basic/general blood pressure estimation model to generate the personal static blood pressure estimation model based on the reference measurement result and PPG waveform signal both corresponding to the linear state.

For an example of linear state (static blood pressure), the device 200 may assume or require that the specific user operates the reference sphygmomanometer to measure blood pressure in the morning after the user gets up for a few minutes or at the night before the user goes to bed, and the processing circuit 215 can control the PPG sensor 210 to detect the physiological characteristics of the specific user for one time to generate one PPG waveform signal after the user gets up for a few minutes or before the user goes to bed. In this example, it is not required for the device 200 to ask or command the specific user to operate the reference sphygmomanometer; the device 200 receives the reference measurement result of the reference sphygmomanometer and uses the PPG sensor 210 to perform physiological characteristics detection merely after the user gets up for a few minutes or before the user goes to bed. In other examples, the device 200 can be designed to ask or command the specific user to operate the reference sphygmomanometer. This is not intended to be a limitation.

Alternatively, if the reference measurement result received by the receiving unit 205 is measured and obtained when the change of blood pressure of specific user is at the non-linear state, the PPG sensor 210 is activated to detect the physiological characteristics of the specific user for one time to generate one PPG waveform signal when the change of blood pressure of specific user is at the same non-linear state, so that the processing circuit 215 can adjust corresponding parameter factor(s) of the basic/general blood pressure estimation model to generate the personal dynamic blood pressure estimation model based on the reference measurement result and PPG waveform signal both corresponding to the non-linear state.

For an example of non-linear state (dynamic blood pressure), the device 200 may assume or require that the specific user operates the reference sphygmomanometer to measure dynamic blood pressure after exercise to know his/her dynamic blood pressure when he/she is exercising, and the processing circuit 215 can control the PPG sensor 210 to detect the physiological characteristics of the specific user for one time to generate one PPG waveform signal after the user exercises. In this example, it is not required for the device 200 to ask or command the specific user to operate the reference sphygmomanometer; the device 200 receives the reference measurement result of the reference sphygmomanometer and uses the PPG sensor 210 to perform physiological characteristics detection merely after the user exercise. In other examples, the device 200 can be designed to ask or command the specific user to operate the reference sphygmomanometer. This is not intended to be a limitation.

In practice, the electronic device 200 may be designed to have multiple different default settings such as a morning mode setting, a night mode setting, and/or an exercise mode setting (e.g. jogging, swimming, or others), and can be implemented using an interactive human-machine interface device which can receive/accept input of the specific user and send a signal to ask/command the specific user to perform an action or behavior.

For instance, the specific user may select the morning mode setting or night mode setting, and the device 200 is arranged to send a signal to ask or command the specific user to measure the static blood pressure by using the reference sphygmomanometer when the specific user gets up for a few minutes or before the specific user goes to bed; in other times, the device 200 does not send a signal to ask or command the specific user to measure the static blood pressure. The processor 215 of device 200 can be arranged to send a signal to instruct the specific user to input the values of measured static blood pressure which can be received by the receiving unit 205. The processor 215 can be arranged to control the PPG sensor 210 to detect the physiological characteristics of the specific user to generate PPG waveform signal(s) at the same time or before/after the specific user inputs the values of measured static blood pressure. Thus, the processor 215 can obtain/calculate the estimation result based on the PPG waveform signal(s) wherein the obtained reference measurement result and the estimation result both correspond to an identical/similar user behavior or action. In other words, the obtained reference measurement result and the calculated estimation result both are associated with the linear state for the blood pressure of the specific user.

Also, the specific user may select the exercise mode setting, and the device 200 is arranged to send a signal to ask or command the specific user to measure the dynamic blood pressure by using the reference sphygmomanometer after/when the specific user does an exercise; the device 200 does not send a signal to ask or command the specific user to measure the dynamic blood pressure by using the reference sphygmomanometer if detecting that the specific user is not exercising. The processor 215 of device 200 can be arranged to send a signal to instruct the specific user to input the values of measured dynamic blood pressure which can be received by the receiving unit 205. The processor 215 can be arranged to control the PPG sensor 210 to detect the physiological characteristics of the specific user to generate PPG waveform signal(s) when/after the specific user inputs the values of measured dynamic blood pressure. Thus, the processor 215 can obtain/calculate the estimation result based on the PPG waveform signal(s) wherein the obtained reference measurement result and the estimation result both correspond to an identical/similar user behavior or action. In other words, the obtained reference measurement result and the calculated estimation result both are associated with the non-linear state for the blood pressure of the specific user.

In other embodiments, the processor 215 can be also arranged to control the PPG sensor 210 to detect the physiological characteristics of the specific user to generate PPG waveform signal (s) by the specific user's control or when the specific user determines to use the estimation of PPG sensor 210. For example, the specific user may select an indoor mode setting of device 200 which corresponds to the linear state of blood pressure estimation, and the specific user may determine when and whether to activate/trigger the PPG sensor 210 to generate PPG waveform signal(s) if the specific user considers some timing is appropriate to estimate the static blood pressure. Similarly, the specific user may select the exercise mode setting corresponding to the non-linear state of blood pressure estimation, and the specific user may determine when and whether to activate/trigger the PPG sensor 210 to generate PPG waveform signal (s) if the specific user considers some timing is appropriate to estimate the dynamic blood pressure.

Further, the device 200 may be designed to be externally and electrically connected to the reference sphygmomanometer via wired/wireless communication, and the receiving unit 205 may be configured to directly receive the reference measurement result from the reference sphygmomanometer without instructing the user to input such measurement result. That is, the device 200 may immediately receive the reference measurement result from the reference sphygmomanometer after the user operates the sphygmomanometer. Then, the device 200 may automatically activate or trigger the PPG sensor 210 to detect the physiological characteristics of the specific user to generate PPG waveform signal(s). In other words, the device 200 can be designed to merely ask/instruct the specific user to use the reference sphygmomanometer, and then automatically receive the reference measurement result and trigger the PPG sensor 210 to generate PPG waveform signal(s).

In addition, for automatically generating a PPG waveform signal for static blood pressure estimation, the device 200 may incorporate with a sleep monitor function which can be used to detect when the user falls asleep and when the user gets up. When detecting the specific user falls asleep or gets up, the processor 215 controls the PPG sensor 210 to automatically detect the physiological characteristics of the specific user to generate a PPG waveform signal.

In Step 120A and Step 120B, for generating personal static and dynamic blood pressure models, the processing circuit 215 is arranged to calculate and obtain an estimation result of the static blood pressure of the specific user according to a generated PPG waveform signal at linear state and to obtain an estimation result of the dynamic blood pressure of the specific user according to a generated PPG waveform signal at non-linear state. In practice, for generating an estimation result, the processing circuit 215 may calculate and obtain the estimation result of blood pressure (either static or dynamic) of the specific user based on an interval between a major peak and a second peak of one PPG waveform signal. The second peak means a reflective wave. However, this is not meant to be a limitation. The processing circuit may perform calculation based on other algorithms and the PPG waveform signal to generate an estimation result.

In Step 125A and Step 125B, the processing circuit 215 is arranged to adjust two sets of parameter factor(s) of the basic/general blood pressure estimation model used by the PPG sensor 210 by comparing the at least two reference measurement results with at least two estimation results of blood pressure respectively, to generate the personal static and dynamic blood pressure estimation models dedicated for the specific user. For example, each reference measurement result has a reference value of systolic blood pressure and a reference value of diastolic blood pressure, and each estimation result of blood pressure has an estimation value of systolic blood pressure and an estimation value of diastolic blood pressure. For instance, the device 200 is arranged to estimate and obtain the estimation value of systolic blood pressure and estimation value of diastolic blood pressure based on waveform component intervals of the generated PPG waveform signal.

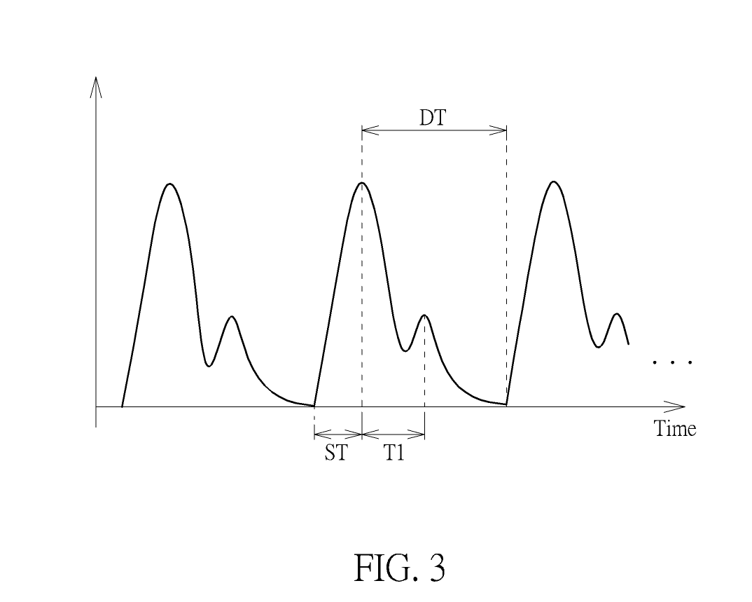

Refer to FIG. 3, which is a diagram showing an example of one PPG waveform signal. As shown in FIG. 3, each repeated waveform component of the PPG waveform signal has three different time intervals ST, DT, and T1. ST means the time interval between the start of the repeated waveform component and the maximum value (i.e. the major peak) of the repeated waveform component. DT means the time interval between the end of the repeated waveform component and the maximum value of the repeated waveform component. T1 means the time interval between the maximum value of the repeated waveform component and the second maximum value (i.e. second peak) of the repeated waveform component.

The basic/general blood pressure estimation model can be represented by the following equations:

ESBP=A1×DT+A2

EDBP=B1×T1+B2

wherein A1, B1, A2, and B2 are basic/general parameter factors, and ESBP and EDBP respectively indicate the estimation values of systolic blood pressure and diastolic blood pressure to be calculated. For example, A1 is equal to −0.095, and B1 is equal to −0.344. A2 is equal to 188.581, and B2 is equal to 174.308. However, this is not intended to be a limitation. The basic/general parameter factors may be configured as different values in other examples.

The processing circuit 215 is arranged to compare the reference value of systolic blood pressure with the estimation value of systolic blood pressure (i.e. ESBP) to calculate a difference which is used as a reference (a regulating/calibration parameter) to regulate or adjust the parameter factor(s) A1 and/or A2. Also, the processing circuit 215 is arranged to compare the reference value of diastolic blood pressure with the estimation value of diastolic blood pressure (i.e. EDBP) to calculate a difference which is used as a reference (another regulating/calibration parameter) to regulate or adjust the parameter factor(s) B1 and/or B2. By respectively perform comparison for static blood pressure and dynamic blood pressure, the device 200 can calculate and obtain the personal blood pressure estimation model (personal static and dynamic blood pressure estimation models) dedicated for a specific user. Based on the personal blood pressure estimation model of device 200, the specific user or person can use the device 200 to automatically estimate and derive more accurate values of his/her blood pressure no matter what behavior or action the user or person is doing now.

Further, the device 200 may be arranged to generate a personal blood pressure estimation model having a set of parameter factors only for systolic blood pressure or only for diastolic blood pressure in response to a particular user's requirement or control. For example, the particular user may measure the reference value of only systolic blood pressure or only diastolic blood pressure by using the reference sphygmomanometer, and the device 200 can be arranged to calculate and adjust the parameter factors only for systolic blood pressure or only for diastolic blood pressure to form the personal blood pressure estimation model dedicated to the particular user.

Additionally, in other embodiments, the device 200 can more precisely define or identify the linear and non-linear states for the change of blood pressure of the specific user according to the heart rate of the specific user, so as to generate and obtain more accurate personal blood pressure estimation model. The device 200 is capable of precisely detecting or measuring a current value, a minimum value, and a maximum value of the specific user's heart rate around all day. For example, the device 200 can be implemented as a wearable electronic device which can be arranged to automatically detecting the specific user's heart rate so as to obtain the minimum value and maximum value of heart rate. The minimum value of heart rate can be defined as the value of specific user's rest heart rate corresponding to the steady state of specific user's blood pressure. So, the device 200 can derive a heart rate reserve percentage P according to the following equation:

$$P = \frac{( E - R )}{( MAX - R )} \times 100 \%$$

wherein parameter R indicates the value of rest heart rate of the specific user, parameter E indicates a currently measured value of heart rate of the specific user, parameter MAX indicates the maximum value of heart rate of the specific user. It should be noted that the maximum value of heart rate of the specific user can be inputted or modified by the specific user in other embodiments.

A steady state for the change of blood pressure means that the heart rate reserve percentage currently measured is equal to zero. For example, the specific user at the steady state may mean that the specific user takes a rest for a few minutes. The device 200 is to define/configure a range of heart rate reserve percentage from zero to a first percentage value such as 10% as the linear state of blood pressure for the specific user, and to define/configure another range of heart rate reserve percentage from the first percentage value such as 10% to a second percentage value such as 100% as the non-linear state of blood pressure for the specific user. For example, the specific user is walking around a room (but not limited), and the heart rate reserve percentage currently measured may be at the linear state. The specific user is jogging outdoors, and the heart rate reserve percentage currently measured may be at the non-linear state.

The device 200 is arranged to estimate the current value of the specific user's heart rate by using PPG sensor 210, and then the processing circuit 215 is arranged to calculate or derive the value of heart rate reserve percentage currently measured. Thus, the processing circuit 215 can accurately determine that the change of specific user's blood pressure is at the linear state or non-linear state. If the processing circuit 215 determines that the change of specific user's blood pressure is at the linear state, the processing circuit 215 is arranged to compare the reference value of static blood pressure measured by the reference sphygmomanometer with the estimation value of static blood pressure calculated based on information of PPG sensor 210, to adjust a set of corresponding parameter factor(s) of the basic/general blood pressure estimation model so as to finally generate the personal static blood pressure estimation model.

Similarly, if the processing circuit 215 determines that the change of specific user's blood pressure is at the non-linear state, the processing circuit 215 is arranged to compare the reference value of dynamic blood pressure measured by the reference sphygmomanometer with the estimation value of dynamic blood pressure calculated based on information of PPG sensor 210, to adjust another set of corresponding parameter factor(s) of the basic/general blood pressure estimation model so as to finally generate the personal dynamic blood pressure estimation model.

Based on the information of heart rate reserve percentage of the specific user, the device 200 can precisely distinguish the linear state from the non-linear state for the change of specific user's blood pressure no matter what actions/behavior the specific user does. Thus, a more accurate personal blood pressure estimation model having static and dynamic estimation models can be obtained.

Further, it should be noted that the electronic device 200 can be also arranged to generate and obtain merely the personal static blood pressure estimation for the specific user or generate and obtain merely the personal dynamic blood pressure estimation for the specific user. FIG. 1 shows a preferred embodiment. This is not intended to be a limitation.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method for establishing a personal blood pressure estimation model dedicated for a specific user, comprising:
receiving a first reference measurement result of a reference sphygmomanometer;
using a photoplethysmogram sensor to measure a blood pressure of the specific user to generate a first photoplethysmogram (PPG) signal;
calculating a first estimation result of the blood pressure of the specific user according to the first PPG signal;
generating a first regulating parameter by comparing the first reference measurement result with the first estimation result; and
establishing the personal blood pressure estimation model by using the first regulating parameter to adjust a set of parameter factor(s) of a basic blood pressure estimation model.

2. The method of claim 1, wherein the first reference measurement result and the first estimation result correspond to an identical state corresponding to a change of the blood pressure of the specific user.

3. The method of claim 2, wherein the identical state is one of a linear state and a non-linear state; the linear state indicates that a value of the blood pressure of the specific user linearly changes with a change of a heart rate of the specific user, and the non-linear state indicates that the value of the blood pressure of the specific user non-linearly changes with the change of the heart rate of the specific user.

4. The method of claim 2, further comprising:
detecting a heart rate reserve percentage of the specific user; and
configuring a range of the heart rate percentage to be associated with the identical state;
wherein the first reference measurement result and the first estimation result are generated and obtained when the detected heart rate reserve percentage falls within the range.

5. The method of claim 1, further comprising:
sending a signal to instruct the specific user to operate the reference sphygmomanometer before receiving the first reference measurement result of the reference sphygmomanometer.

6. The method of claim 1, further comprising:
receiving a second reference measurement result of the reference sphygmomanometer;
using the PPG sensor to measure the blood pressure of the specific user to generate a second PPG signal;
calculating a second estimation result of the blood pressure of the specific user according to the second PPG signal;
generating a second regulating parameter by comparing the second reference measurement result with the second estimation result; and
establishing the personal blood pressure estimation model by further using the second regulating parameter to adjust another set of parameter factor(s) of the basic blood pressure estimation model.

7. The method of claim 6, wherein the first reference measurement result and the first estimation result correspond to a first state corresponding to a change of the blood pressure of the specific user, and the second reference measurement result and the second estimation result correspond to a second state corresponding to the change of the blood pressure of the specific user.

8. The method of claim 7, wherein the first state is a linear state which indicates that a value of the blood pressure of the specific user linearly changes with a change of a heart rate of the specific user, and the second state is a non-linear state which indicates that the value of the blood pressure of the specific user non-linearly changes with the change of the heart rate of the specific user.

9. The method of claim 8, wherein the personal blood pressure estimation model comprises a personal static blood pressure estimation model and a personal dynamic blood pressure estimation model.

10. The method of claim 8, further comprising:
detecting a heart rate reserve percentage of the specific user; and
configuring a first range of the heart rate percentage to be associated with the linear state and a second range of the heart rate percentage to be associated with the non-linear state;
wherein the first reference measurement result and the first estimation result are generated and obtained when the detected heart rate reserve percentage falls within the first range, and the first reference measurement result and the first estimation result are generated and obtained when the detected heart rate reserve percentage falls within the second range.

11. An electronic device for establishing a personal blood pressure estimation model dedicated for a specific user, comprising:
a receiving unit, configured to receive a first reference measurement result of a reference sphygmomanometer;
a photoplethysmogram (PPG) sensor, configured to measure a blood pressure of the specific user to generate a first PPG signal; and
a processing circuit, coupled to the receiving unit and the PPG sensor, configured to:
calculate a first estimation result of the blood pressure of the specific user according to the first PPG signal;
generate a first regulating parameter by comparing the first reference measurement result with the first estimation result; and
establish the personal blood pressure estimation model by using the first regulating parameter to adjust a set of parameter factor(s) of a basic blood pressure estimation model.

12. The electronic device of claim 11, wherein the first reference measurement result and the first estimation result correspond to an identical state corresponding to a change of the blood pressure of the specific user.

13. The electronic device of claim 12, wherein the identical state is one of a linear state and a non-linear state; the linear state indicates that a value of the blood pressure of the specific user linearly changes with a change of a heart rate of the specific user, and the non-linear state indicates that the value of the blood pressure of the specific user non-linearly changes with the change of the heart rate of the specific user.

14. The electronic device of claim 12, wherein the processing circuit controls the PPG sensor to detect a heart rate reserve percentage of the specific user; the processing circuit configures a range of the heart rate percentage to be associated with the identical state; and, the first reference measurement result and the first estimation result are generated and obtained when the processing circuit decides that the detected heart rate reserve percentage falls within the range.

15. The electronic device of claim 11 is an interactive human-machine interface device which is configured to send a signal to instruct the specific user to operate the reference sphygmomanometer before receiving the first reference measurement result of the reference sphygmomanometer.

16. The electronic device of claim 11, wherein the receiving unit is arranged to receive a second reference measurement result of the reference sphygmomanometer; the PPG sensor is used to measure the blood pressure of the specific user to generate a second PPG signal; and, the processing circuit is arranged for: calculating a second estimation result of the blood pressure of the specific user according to the second PPG signal; generating a second regulating parameter by comparing the second reference measurement result with the second estimation result; and, establishing the personal blood pressure estimation model by further using the second regulating parameter to adjust another set of parameter factor(s) of the basic blood pressure estimation model.

17. The electronic device of claim 16, wherein the first reference measurement result and the first estimation result correspond to a first state corresponding to a change of the blood pressure of the specific user, and the second reference measurement result and the second estimation result correspond to a second state corresponding to the change of the blood pressure of the specific user.

18. The electronic device of claim 17, wherein the first state is a linear state which indicates that a value of the blood pressure of the specific user linearly changes with a change of a heart rate of the specific user, and the second state is a non-linear state which indicates that the value of the blood pressure of the specific user non-linearly changes with the change of the heart rate of the specific user.

19. The electronic device of claim 18, wherein the personal blood pressure estimation model comprises a personal static blood pressure estimation model and a personal dynamic blood pressure estimation model.

20. The electronic device of claim 18, wherein the processing circuit is arranged to control the PPG sensor to detect a heart rate reserve percentage of the specific user; and the processing circuit configures a first range of the heart rate percentage to be associated with the linear state and a second range of the heart rate percentage to be associated with the non-linear state; the first reference measurement result and the first estimation result are generated and obtained when the processing circuit decides that the detected heart rate reserve percentage falls within the first range, and the first reference measurement result and the first estimation result are generated and obtained when the processing circuit decides that the detected heart rate reserve percentage falls within the second range.
